(19) United States
(12) Patent Application Publication
(10) Pub. No.: US 2002/0081632 A1
(43) Pub. Date: Jun. 27, 2002

(54) Assays to assess conformational changes of integrins induced by ligand binding based on electrophoretic mobility

(76) Inventors: Richard Wynn, Bear, DE (US); Nina I. Zolotararjova, Wilmington, DE (US)

Correspondence Address: BRISTOL-MYERS SQUIBB PHARMA COMPANY, PATENT DEPARTMENT, P.O. BOX 4000, PRINCETON, NJ, 08543-4000, US

(21) Appl. No.: 09/994,493

(22) Filed: Nov. 26, 2001

Related U.S. Application Data
(63) Continuation of application No. 09/487,404, filed on Jan. 26, 2000, now abandoned.
(60) Provisional application No. 60/117,257, filed on Jan. 26, 1999.

(51) Int. Cl. G01N 33/53; G01N 33/567; C02F 1/469

(52) U.S. Cl. 435/7.2; 204/456

(57) ABSTRACT

This invention relates to electrophoretic assays useful for the detection of conformational changes in integrin structure upon binding of antagonists, agonists or ligands. Such assays are useful to identify drug candidate compounds that may have a high potential for side reactions when administered in patients.

### FIELD OF THE INVENTION

This invention relates to electrophoretic assays useful for the detection of conformational changes in integrin structure upon binding of antagonists, agonists or ligands. Such assays are useful to identify drug candidate compounds that may have a high potential for side reactions when administered in patients.

### BACKGROUND OF THE INVENTION

Thromboembolic diseases, including stable and unstable angina pectoris, myocardial infarction, stroke and lung embolism, are the major cause of disability and mortality in most developed countries. Recently, therapeutic strategies aimed at interfering with the binding of ligands to the GPIIb/IIIa integrin have been explored to treat these patient groups. Platelet GPIIb/IIIa is the main platelet receptor for fibrinogen and other adhesive glycoproteins, including fibronectin, vitronectin and von Willebrand factor. Interference of ligand binding with this receptor has been proven beneficial in animal models of thromboembolic disease (Coller, B. S. GPIIb/IIIa Antagonists: Pathophysiologic and Therapeutic Insights From Studies of C7E3 FAB. Thromb. Haemost. 78: 1, 730-735, 1997), and in limited studies involving human subjects (White, H. D. Unmet Therapeutic Needs in the Management of Acute Ixchemia. Am. J. Cardiol. 80: 4A, 2B-10B, 1997; Tcheng, J. E. Glycoprotein IIb/IIIa Receptor Inhibitors: Putting EPIC, IMPACT II, RESTORE, and EPILOG Trials Into Perspective. Am. J. Cardiol. 78: 3A, 35-40, 1996).

A number of cell surface receptor proteins, referred to as integrins or adhesion protein receptors, have been identified which bind to extracellular matrix ligands or other cell adhesion protein ligands thereby mediating cell-cell and cell-matrix adhesion processes. The integrins are encoded by genes belonging to a gene superfamily and are typically composed of heterodimeric transmembrane proteins containing α- and β-subunits. Integrin subfamilies contain a common β-subunit combined with different α-subunits to form adhesion protein receptors with different specificities. In addition to GPIIb/IIIa, a number of other integrin cell surface receptors have been identified. For example, members of the β1 subfamily, α4β1 and α5β1, have been implicated in various inflammatory processes, including rheumatoid arthritis, allergy, asthma and autoimmune disorders.

The integrin GPIIb/IIIa, also referred to as the platelet fibrinogen receptor, is the membrane protein mediating platelet aggregation. GPIIb/IIIa in activated platelets is known to bind four soluble RGD containing adhesive proteins, namely fibrinogen, von Willebrand factor, fibronectin, and vitronectin. The term “RGD” -refers to the amino acid sequence Arg-Gly-Asp. The binding of fibrinogen and von Willebrand factor to GPIIb/IIIa causes platelets to aggregate. The binding of fibrinogen is mediated in part by the RGD recognition sequence which is common to the adhesive proteins that bind GPIIb/IIIa. RGD-peptidomimetic GPIIb/IIIa antagonist compounds are known to block fibrinogen binding and prevent platelet aggregation and the formation of platelet thrombi. GPIIb/IIIa antagonists represent an important new approach for anti-platelet therapy for the treatment of thromboembolic disorders.

Approximately 1% of individuals receiving such antagonists develop thrombocytopenia, a potentially life threatening complication (Berkowitz, S. D., Harrington, R. A., Rund, M. M., and Tcheng, J. E. Acute Profound Thrombocytopenia After C7E3 FAB (abciximab) Therapy. Circulation 95: 809-813, 1997). This side effect severely compromises the development of GPIIb/IIIa antagonists as a viable treatment strategy. Thrombocytopenia most likely arises from the presence and/or development of drug dependent antibodies (DDABs) to neoepitopes induced by the binding of antagonists to GPIIb/IIIa. The presence of neoepitopes suggests significant conformational change(s) in the receptor upon antagonist binding.

An assay to detect changes in receptor conformation after antagonist, agonist or ligand binding would be useful to determine which compounds have significant potential for inducing thrombocytopenia or other side effects when administered in vivo. Such an assay could be used in the development of safe efficacious compounds and for accessing the safety of drug candidate compounds.

### SUMMARY OF THE INVENTION

This invention relates to assays useful for the detection of conformational changes in integrin structure upon binding of antagonists, agonists or ligands.

An object of the present invention provides an assay that detects conformational changes in the integrin's structure.

A preferred embodiment of the present invention provides that the cell surface adhesion receptor is glycoprotein IIb/IIIa (GPIIb/IIIa).

### BRIEF DESCRIPTION OF THE DRAWINGS

FIG. 1. Electrophoretic mobility changes in a denaturing PAGE system for purified GPIIb/IIIa pre-incubated with an antagonist. Purified GPIIb/IIIa was incubated with either water (−lane) or excess compound A (+lane) in 0.1% Triton X-100 (v/v), 20 mM Tris-HCl, 1 mM MgCl₂, 1 mM CaCl₂, 150 mM NaCl, pH 7.5 for 1 hour at 37° C. An equal volume of 2×SDS buffer was added and electrophoresis was carried out using Novex 10-20% gradient gels under manufacturers protocols.

FIG. 2. Electrophoretic mobility changes in a native PAGE system for purified GPIIb/IIIa pre-incubated with an antagonist. Purified GPIIb/IIIa was incubated with either water (−lane) or excess compound A (+lane) in 0.1% Triton X-100 (v/v), 20 mM Tris-HCl, 1 mM MgCl₂, 1 mM CaCl₂, 150 mM NaCl, pH 7.5 for 1 hour at 37° C. Native gels were poured with 4% acrylamide in the stacking gels and 6.0% acrylamide separating gels using 20 mM Tris-HCl, 0.1% Triton X-100 (v/v) as a buffer. Electrophoresis was carried our for 4 hr. at 100 v.

FIG. 3. Electrophoretic mobility changes in a denaturing SDS PAGE system for platelet GPIIb/IIIa pre-incubated with an antagonist. Fresh platelets were incubated with either water (lane 1) or increasing amounts of compound A (lanes 2-5) in 20 mM Tris-HCl, 150 mM NaCl, 1 mM MgCl₂, 1 mM CaCl₂, 0.5 mM AEBSF, 100 μM E-64, 20 μM Leupeptin, pH 7.5 for 1 hour at 37° C. An equal volume of 2×SDS buffer was added and electrophoresis was carried out using Novex 10-20% gradient gels under manufacturers protocols. Proteins were blotted onto PVDF membranes and Western analysis was carried out with a mixture of monoclonal antibodies specific for the IIb and IIIa peptide chains (panel A) or JK094 (panel B).

FIG. 4. Electrophoretic mobility changes in a denaturing system for purified GPαᵥβ₃ pre-incubated with an antagonist. Purified GPᵥβ₃ was incubated with either water (lane 2) or excess compound B (lane 3) in 0.1% Triton X-100 (v/v), 20 mM Tris-HCl, 1 miM MgCl₂, 1 mM CaCl₂, 150 mM NaCl, pH 7.5 for 1 hour at 37° C. An equal volume of 2×SDS buffer was added and electrophoresis was carried out using Novex 10-20% gradient gels under manufacturers protocols.

### DETAILED DESCRIPTION OF THE INVENTION

An embodiment of the present invention provides a method for identifying cell surface adhesion receptor antagonists, agonists or ligands that have a high potential of side effects, comprising:

(a) contact a compound which binds to the cell surface adhesion receptor with the cell surface adhesion receptor;

(b) run in a first lane of an electrophoresis gel the resulting bound compound:cell surface adhesion receptor;

(c) run in a second lane of the electrophoresis gel the native cell surface adhesion receptor;

(d) visualize the protein bands; and

(e) compare the mobility and banding patterns of the first and second lanes to determine if a conformational change has occurred.

A preferred embodiment provides the cell surface adhesion receptor is an integrin.

A more preferred embodiment provides the cell surface adhesion receptor is glycoprotein IIb/IIIa (GPIIb/IIIa).

Another preferred embodiment provides the electrophoresis gel is a native electrophoresis format.

Another preferred embodiment provides the electrophoresis gel is a denaturing electrophoresis format.

Another preferred embodiment provides the electrophoresis gel is a slab electrophoresis format.

Another preferred embodiment provides the electrophoresis gel is a capillary electrophoresis format.

Another preferred embodiment provides the protein bands are visualized by normal protein staining techniques, including but not limited to, Coomassie staining, silver staining and fluorescent staining.

Another preferred embodiment provides the protein bands are visualized by Western Blot techniques.

The term “PAGE”, as used herein refers to polyacrylamide gel electrophoresis; the term “SDS-PAGE” refers to sodium dodecylsulfate-polyacrylamide gel electrophorsis.

The term “mobility ”, as used herein, refers to the distance the band travels in an electrophoresis gel. The term “banding pattern”, as used herein, refers to whether one, or more than one, band appears in the electrophoresis gel. By observing the mobility and banding patterns of a bound compound:GPIIb/IIIa with native GPIIb/IIIa, one skilled in the art can determine if a conformational change has occurred. If such a change is observed, one can predict that the compound has a high potential for side effects. This assay can be used to screen chemical drug candidates prior to administration to patients.

The term “integrin” as used herein refers to any of the many cell surface receptor proteins, also referred to as adhesion protein receptors, which have been identified which bind to extracellular matrix ligands or other cell adhesion protein ligands thereby mediating cell-cell and cell-matrix adhesion processes. The integrins are encoded by genes belonging to a gene superfamily and are typically composed of heterodimeric transmembrane glycoproteins containing α- and β-subunits. Integrin subfamilies contain a common β-subunit combined with different α-subunits to form adhesion protein receptors with different specificities.

The integrin glycoprotein IIb/IIIa (referred to herein as GPIIb/IIIa or IIb/IIIa or the fibrinogen receptor) is the membrane protein mediating platelet aggregation. GPIIb/IIIa in activated platelets is known to bind four soluble RGD-containing adhesive proteins, namely fibrinogen, von Willebrand factor, fibronectin, and vitronectin. In addition to GPIIb/IIIa, a number of other integrin cell surface receptors have been identified, for example, αvβ3, α4β1 and α5β1.

The integrins used in the present assays may be obtained from a non-recombinant source (as described in Example 1 for GPIIb/IIIa) or from a recombinant source using a recombinant expression vector encoding the desired integrin and a host expression system. In the case of the recombinant integrin, such integrin may differ from the non-recombinant or native form of the integrin in being a fragment and/or an altered, fused or mutant form of the non-recombinant or native form of the integrin.

The term “agonist” as used herein, refers to an agent (including but not limited to proteins, peptides, peptideomimetic compounds and other small molecule compounds) capable of stimulating a biological response by occupying cell receptors.

The term “ligand” as used herein, refers to a small molecule (including but not limited to proteins, peptides, peptideomimetic compounds and other small molecule compounds) that binds specifically to a larger molecule.

The term “integrin antagonists” as referred to herein (also referred to herein as integrin inhibitors) includes compounds (including but not limited to proteins, peptides, peptideomimetic compounds and other small molecule compounds) which act as inhibitors of the binding of the integrin protein to endogenous protein ligands of such integrin. Preferred integrin inhibitors used in the present invention are RGD-peptidomimetic compounds. As used herein, the term “RGD-peptidomimetic compounds” refers to chemical compounds which bind to the RGD-binding region of the integrin and which block RGD-mediated binding of one or more adhesive proteins to such integrin. Preferred in the present invention are antagonists of the integrin GPIIb/IIIa.

Representative integrin antagonist compounds, including GPIIb/IIIa antagonists are disclosed in the following patents and patent applications, which are incorporated herein by reference: PCT Patent Application 95/14683; PCT Patent Application 95/32710; U.S. Pat. No. 5,334,596; U.S. Pat. No. 5,276,049; U.S. Pat. No. 5,281,585; European Patent Application 478,328; European Patent Application 478,363; European Patent Application 512,831; PCT Patent Application 94/08577; PCT Patent Application 94/08962; PCT Patent Application 94/18981; PCT Patent Application 93/16697; Canada Patent Application 2,075,590; PCT Patent Application 93/18057; European Patent Application 445,796; Canada Patent Application 2,093,770; Canada Patent Application 2,094,773; Canada Patent Application 2,101,179; Canada Patent Application 2,074,685; Canada Patent Application 2,094,964; Canada Patent Application 2,105,934; Canada Patent Application 2,114,178; Canada Patent Application 2,116,068; European Patent Application 513,810; PCT Patent Application 95/06038; European Patent Application 381,033; PCT Patent Application 93/07867; and PCT Patent Application 94/02472.

GPIIb/IIIa antagonists useful as positive controls in assays of the present invention are the compounds A-D listed below, and salt forms, prodrug forms and metabolites thereof.

The preparation of 2(S)-[(n-butoxycarbonyl)amino]-3-[[[3-[4-(aminoiminomethyl)phenyl]isoxazolin-5(R)-yl]methylcarbonyl]amino]propionic acid or its methyl ester is disclosed in PCT Patent Application Publication No. WO 95/14683, herein referred to as “Compound A”.

The preparation of 2(S)-[[(3,5-dimethylisoxazol-4-yl) sulfonyl]amino]-3-[[[3-[4-(aminoiminomethly) phenyl]isoxazolin-5(R)-yl]methylcarbonyl]amino]propionic acid is disclosed in PCT Patent Application Publication Number WO 96/37482, published Nov. 28, 1996, herein referred to as “Compound B”.

The preparation of 2(S)-[(4-methylphenylsulfonyl) amino]-3-[[[5,6,7,8-tetrahydro-4-oxo-5-[2-(piperidin-4-yl)ethyl ]-4H-pyrazolo-[1,5-a] [1,4] diazepin-2-yl]carbonyl]amino]propionic acid is disclosed in PCT Patent Application Publication Number WO 94/18981, herein referred to as “Compound C”.

The preparation of 5-[2-(piperdin-4-yl) ethyl]thieno[2,3-b]thiophene-2-N-(3-2(S)-(3-pyridinylsulfonylamino)propionic acid]carboxamide is disclosed in PCT Patent Application Publication No. WO 95/14351, herein referred to as “Compound D”.

The term “JK094” as used herein refers to a chimeric monoclonal antibody specific for the gel-shifted form of GPIIb/IIIa, whose cloning, PCR recombination, production, purification and characterization are disclosed in commonly-owned pending US patent application Ser. No. 09/237061 the contents of which are incorporated herein by reference.

The invention can be further understood by the following examples in which parts and percentages are by weight unless otherwise indicated. It should be understood that these examples, while indicating preferred embodiments of the invention, are given by way of illustration only. From the above discussion and these examples, one skilled in the art can ascertain the essential characteristics of this invention. Preferred embodiments of the invention have been chosen for the purposes of illustration and description but are not intended in any way to restrict the scope of the invention. Preferred embodiments of certain aspects of the invention are shown in the accompanying drawings.

### EXAMPLE 1

### Purification of GPIIb/IIIa Suitable for use in Gel Shift Assay

Outdated platelet concentrates were purchased from Interstate Blood Bank. Platelet concentrates (100 units) were pooled into six 1-liter centrifuge bottles and centrifuged at 300 g for 5 minutes to remove red blood cells. The platelet supernatant was removed and centrifuged at 1800 g for 15 minutes to pellet the platelets, which were subsequently washed three times at 4° C. in wash buffer (20 mM Tris-HCl, 150 mM NaCl, 1 mM EDTA, pH 7.2). The platelet pellet after the last wash was resuspended with 100 mL of lysis buffer (1% Triton X-100 (v/v) , 20 mM Tris-HCl, 1 mM MgCl2, 1 mM CaCl2, 10 uM Leupeptin, 0.5 mM AEBSF, 50 uM E-64, pH 7.4) and shaken for 16 hours at 4° C. The lysed platelets were then centrifuged at 30,000 g to remove membrane cytoskeletons. Lysates were stored at −70° C. until further processing.

Lysate was centrifuged at 30,000 g to remove particles. Concanavalin A Sepharose 4B (capacity of 20 mg glycoprotein/mL gel—Sigma) was poured into 25 mL column. Column was eluted with buffer A (0.1% Triton X-100 (v/v), 20 mM Tris-HCl, 1 mM MgCl2, 1 mM CaCl2, 150 mM NaCl, pH 7.4). Lysates from 50 units of outdated platelet concentrates were adsorbed to the Concanavalin A column at a flow rate of 1 mL/min. at room temperature. The column was washed with 5 bed volumes of buffer A, and the Concanavalin A-retained glycoproteins were eluted with buffer A that contained 100 mM methyl-a-D-mannopyranoside (Sigma) at a flow rate of 0.5 mL/min. and collected into 2 mL fractions. Protein eluted from the Concanavalin A column (50 mL) was dialyzed against buffer A for 18 hrs. at 4° C. Then protein was concentrated to 16 mL by ultrafiltration through YM 100 membranes (Amicon) and further purified on a RGDS-affinity column.

The RGDS-affinity column was prepared by reaction of 15 g of Sepharose 4B activated with 6-aminohexanoic acid N-hydroxysuccinimide ester (Sigma) with 400 mg of RGDS peptide (Bachem) according to manufacturer protocol. Concanavalin A retained protein fraction was applied to the RGDS affinity column (2.3×10 cm) with the flow rate of 0.2 mL/min. The column was washed with buffer A and retained fraction was eluted using a solution of 3 mM RGDS peptide in buffer A and dialyzed extensively against buffer A for 18 hrs. at 4° C. This material is referred to herein as “active GPIIb/IIIa”.

Flow through fractions from RGDS-affinity column were concentrated by ultrafiltration (YM 100) to 8-10 mL and further subjected to size-exclusion chromatography on a Sephacryl S-300 column (3.0×115 cm). The column was eluted at room temperature with buffer A. A flow rate of 100 mL/h was used and 5 mL fractions were collected and analyzed by SDS-PAGE. Fractions containing GPIIb/IIIa was pooled and stored at −70° C. This material is referred to herein as “inactive GPIIb/IIIa”. The terms ‘active’ and ‘inactive’ refer only to binding of the RGDS matrix.

### EXAMPLE 2

### Gel Shift Measurement of Purified GPIIb/IIIa in a Denaturing PAGE System

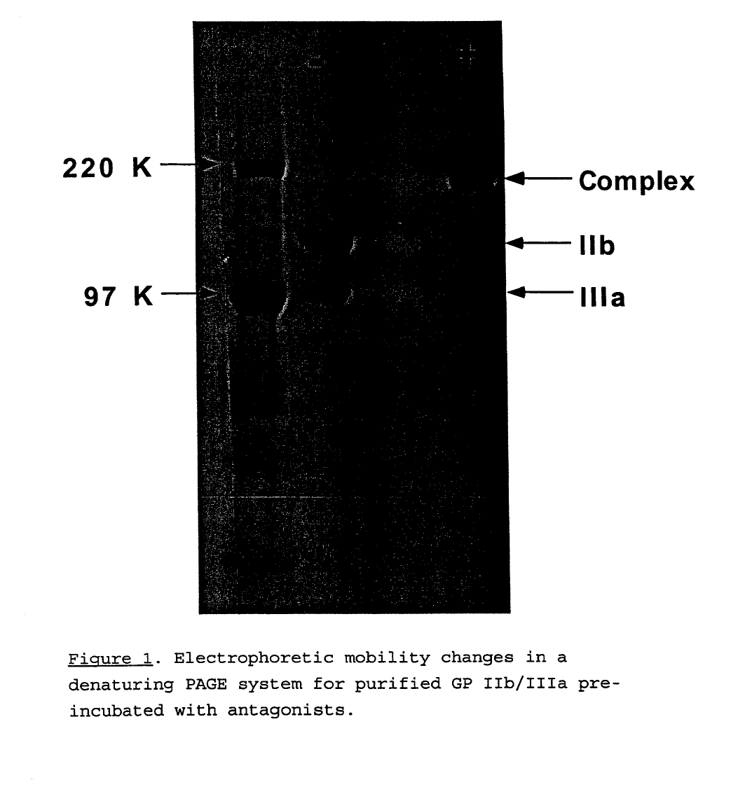

5 μL of purified active or inactive GPIIb/IIIa (1.0 mg/mL) in 0.1% Triton X-100 (v/v), 20 mM Tris-HCl, 1 mM MgCl₂, 1 mM CaCl₂, 150 mM NaCl, pH 7.5 was mixed with 2.5 μL of compound A in water (10 uM) or 2.5 μL of water and incubated for 1 hour at 37° C. 7.5 μL of 2×SDS buffer (126 mM Tris-HCl, 20% glycerol, 4% SDS, 0.005% Bromophenol Blue, pH 6.8) were added and the samples were loaded into 10 well 1 mm 4-20% Tris-Glycine gradient gels from Novex. Electrophoresis was carried out at 185 v for 70 minutes at room temperature using 25 mM Tris, 192 mM Glycine, 0.1% SDS, pH 8.3 as the running buffer. Gels were shaken in 10 mL of deionized water for 5 minutes three times and then stained with 10 mL of Gelcode Blue Coomassie reagent (Pierce) for 1 hr. The gel was then destained in 20 mL of deionized water for 1 hr. typical result is shown in FIG. 1.

### EXAMPLE 3

### Gel Shift Measurement of Purified GPIIb/IIIa in a Native PAGE System

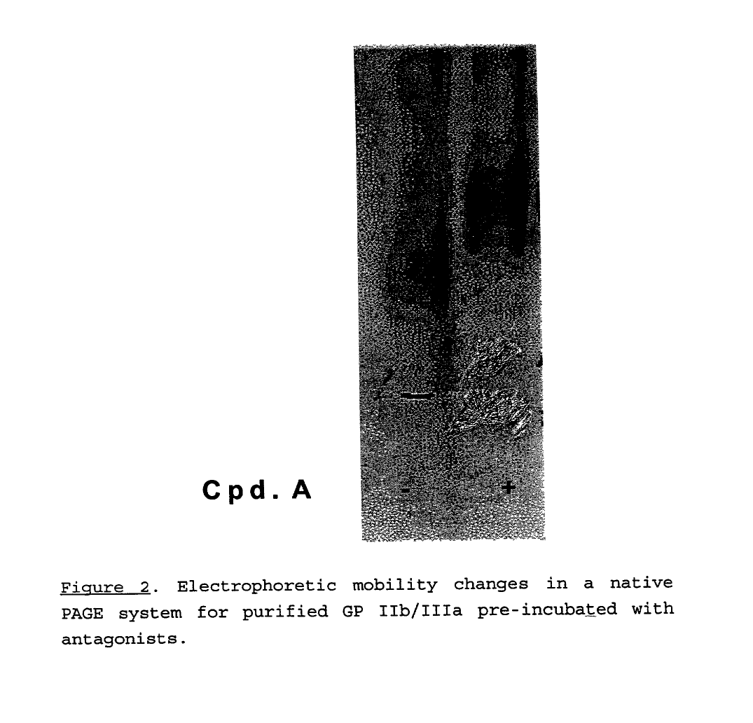

5 μL of purified active or inactive GPIIb/IIIa (1.0 mg/mL) in 0.1% Triton X-100 (v/v), 20 mM Tris-HCl, 1 mM MgCl₂, 1 mM CaCl₂, 150 mM NaCl, pH 7.5 was mixed with 2.5 μL of compound A in water (10 uM) or 2.5 μL of water and incubated for 1 hour at 37° C. 7.5 μL of 25 mM Tris, 192 mM Glycine, 0.1% Triton X-100 (v/v), pH 8.3 was added to each sample. 10 cm×10 cm gels were poured with 4% acrylamide/ 0.2% bisacrylamide in the stacking gels and 6.0% acrylamide/ 0.2% bisacrylamide in the separating gels using 375 mM Tris-HCl, 0.1% Triton X-100 (v/v) as a buffer. Samples were loaded and electrophoresis was carried our for 4 hr. at 100 v at room temperature. Gels were shaken in 10 mL of deionized water for 5 minutes three times and then stained with 10 mL of Gelcode Blue Coomassie reagent (Pierce) for 1 hr. The gel was then destained in 20 mL of deionized water for 1 hr. A typical result is shown in FIG. 2.

### EXAMPLE 4

### Gel Shift Measurement of Platelet GPIIb/IIIa in a Denaturing PAGE System

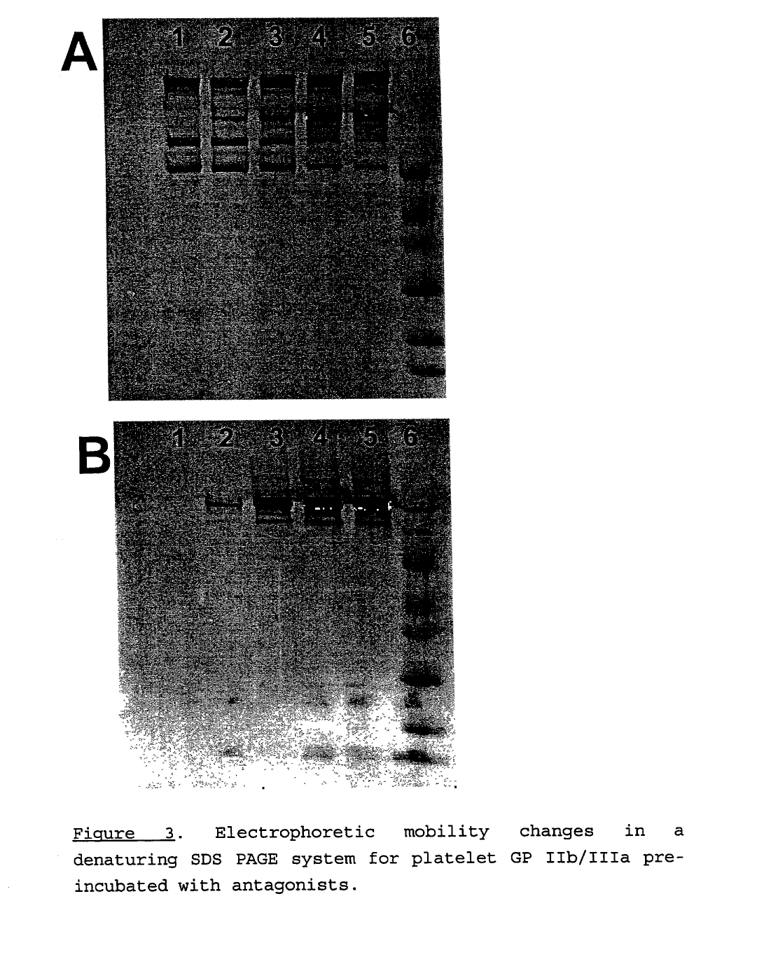

0.25 units of fresh concentrated platelets were obtained from Interstate Blood Bank and washed 3 times with 200 mM Tris-HCl, 150 mM NaCl, 1 mM EDTA, pH 7.2 by repeated pelleting at 5000 rpm. The final pellet was suspended in 0.75 mL 20 mM Tris-HCl, 150 mM NaCl, 1 mM MgCl₂, 1 mM CaCl₂, 0.5 mM AEBSF, 100 uM E-64, 20 uM Leupeptin, pH 7.5. 100 μL of this sample was mixed with 1 μL of compound A at different concentrations or water and incubated at 37° C. for 1 hr. 100 μL of 2×SDS buffer (126 mM Tris-HCl, 20% glycerol, 4% SDS, 0.005% Bromophenol Blue, pH 6.8) were added and the samples were loaded into 10 well 1 mm 4-20% Tris-Glycine gradient gels from Novex. Electrophoresis was carried out at 185 v for 70 minutes at room temperature using 25 mM Tris, 192 mM Glycine, 0.1% SDS, pH 8.3 as the running buffer. Proteins were transferred onto PVDF membranes for 2 hours at 50 v with ice pack cooling using 12 mM Tris, 96 mM Glycine, pH 8.3 as a transfer buffer. PVDF membranes were immersed in blocking solution with shaking for 2 hr. Incubation with primary antibody was carried out overnight at 4° C. The primary antibodies used was either a mixture of the commercially available antibodies SZ21 and SZ22 which target the IIb and IIIa chains, respectively or JK094, a mouse monoclonal antibody specific for the gel-shifted form of GPIIb/IIIa. Detection was carried out with alkaline phosphatase conjugated secondary antibodies. A typical result is shown in FIG. 3.

### EXAMPLE 5

### Gel Shift Measurement of Purified GPαᵥβ₃ in a Denaturing PAGE System

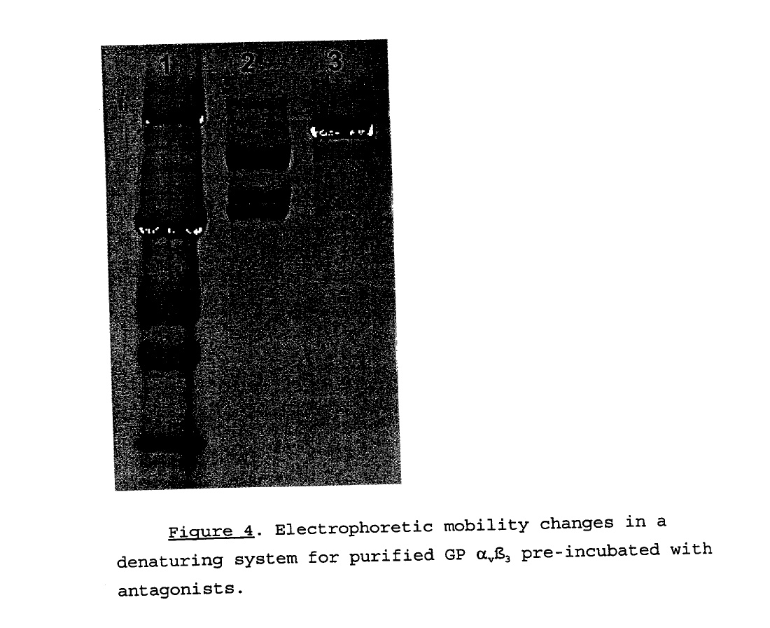

Glycoprotein αvβ₃ (GPαvβ₃) was purchased from Chemicon International, Inc. 5.0 μL of GPαᵥβ₃ (1 mg/mL) in 0.1% Triton X-100 (v/v), 20 mM Tris-HCl, 1 mM MgCl₂, 1 mM CaCl₂, 150 mM NaCl, pH 7.5 was mixed with 2.5 μL of antagonist (1 mM) and incubated at 37° C. for 1 hr. 7.5 μL of 2×SDS buffer (126 mM Tris-HCl, 20% glycerol, 4% SDS, 0.005% Bromophenol Blue, pH 6.8) were added and the samples were loaded into 10 well 1 mm 4-20% Tris-Glycine gradient gels from Novex. Electrophoresis was carried out at 185 v for 70 minutes at room temperature using 25 mM Tris, 192 mM Glycine, 0.1% SDS, pH 8.3 as the running buffer. Gels were shaken in 10 mL of deionized water for 5 minutes three times and then stained with 10 mL of Gelcode Blue Coomassie reagent (Pierce) for 1 hr. The gel was then destained in 20 mL of deionized water for 1 hr. A typical result is shown in FIG. 4.

What is claimed is:

1. A method for identifying cell surface receptor antagonists, agonists or ligands that have a high potential of side effects, comprising:
(a) contact a compound which binds to the cell surface receptor with the cell surface receptor;
(b) run in a first lane of an electrophoresis gel the resulting bound compound:cell surface receptor;
(c) run in a second lane of the electrophoresis gel the native cell surface receptor;
(d) visualize the protein bands; and
(e) compare the mobility and banding patterns of the first and second lanes to determine if a conformational change has occurred.

2. A method according to claim 1 wherein the cell surface receptor is an integrin.

3. A method according to claim 1 wherein the cell surface receptor is glycoprotein IIb/IIIa (GPIIb/IIIa).

4. A method according to claim 1 wherein the electrophoresis gel is a native electrophoresis format.

5. A method according to claim 1 wherein the electrophoresis gel is a denaturing electrophoresis format.

6. A method according to claim 1 wherein the electrophoresis gel is a slab electrophoresis format.

7. A method according to claim 1 wherein the electrophoresis gel is a capillary electrophoresis format.

8. A method according to claim 1 wherein the protein bands are visualized by protein staining techniques.

9. A method according to claim 1 wherein the protein bands are visualized by Western Blot techniques.

10. A method according to claim 1 wherein a third lane is run that contains an antagonist used as an internal positive control selected from the following group:
(a) 2(S)-[(n-butoxycarbonyl)amino]-3-[[[3-[4-(aminoiminomethyl)phenyl]isoxazolin-5(R)-yl]methyl carbonyl]amino]propionic acid or its methyl ester;
(b) 2(S)-[[(3,5-dimethylisoxazol-4-yl)sulfonyl]amino]-3-[[[3-[4-(aminoiminomethly)phenyl]isoxazolin-5(R)-yl]methylcarbonyl]amino]propionic acid;
(c) 2(S)-[(4-methylphenylsulfonyl) amino]-3-[[[5,6,7,8-tetrahydro-4-oxo-5-[2-(piperidin-4-yl)ethyl]-4H-pyrazolo-[1,5-a][1,4]diazepin-2-yl )carbonyl]amino]propionic acid; and
(d) 5-[2-(piperdin-4-yl)ethyl]thieno[2,3-b]thiophene-2-N-(3-2(S)-(3-pyridinylsulfonylamino) propionic acid]carboxamide.
